(19) United States
(12) Patent Application Publication
(10) Pub. No.: US 2023/0081608 A1
(43) Pub. Date: Mar. 16, 2023

(54) APPARATUS AND METHODS FOR MAPPING PERIPHERAL NEUROPATHY

(71) Applicant: Optum, Inc., Minnetonka, MN (US)

(72) Inventors: Jon Kevin Muse, Thompsons Station, TN (US); Gregory J. Boss, Saginaw, MI (US); Ramprasad Anandam Gaddam, Mumbai (IN); Komal Khatri, Cedar Park, TX (US)

(21) Appl. No.: 17/447,627

(22) Filed: Sep. 14, 2021

(51) Int. Cl. A61B 5/00; G06F 3/0487; G06T 11/00; A61N 1/36

(52) U.S. Cl. CPC A61B 5/4041; G06F 3/0487; G06T 11/00; A61B 5/0051; A61B 5/483; A61N 1/36014; A61B 5/6807; A61B 5/743; G06T 2210/41

(57) ABSTRACT

To provide objective indications of the progression of peripheral neuropathy of patients, a transmitter apparatus is utilized to produce reproducible pulse signals to specified locations on the human body to determine a minimum level of stimulation necessary to elicit a consciously detectable sensation through the generation of a series of pulse signals of varying intensities, and the recordation of the number of pulse signals felt by the patient. Moreover, the nerve velocity of the patient is additionally determined for a patient via the transmitter apparatus and a receiver apparatus detecting body-signals passing along a nerve within the patient and corresponding to the pulse signals applied to the patient. The results of these tests are recorded over time, thereby enabling generation of patient-specific models indicative of the progression of the patient's peripheral neuropathy over time.

### BACKGROUND

Peripheral neuropathy is a characterization of a patient's loss of feeling within discrete regions of the patient's body. Practically speaking, this loss of feeling is experienced in terms of a degree of sensory loss—in other words, somewhere along a continuum between complete loss of feeling and no loss of feeling. However, it is generally difficult to obtain an objective indication of the level of sensory loss of a patient, because different patients may describe different levels of feeling loss differently. Even a single patient may describe levels of feeling loss differently on different days, making it difficult or impossible to track the progression of peripheral neuropathy over time for a patient.

Accordingly, a need exists for systems and methods for objectively determining the level of sensory loss experienced by a patient with peripheral neuropathy.

### BRIEF SUMMARY

Embodiments as described herein provide systems and methods for tracking various aspects of peripheral neuropathy over time. For determining an objective indication of the level of conscious, sensory feeling loss of the patient (i.e., the change in the level of feeling that a patient can consciously recognize), various embodiments determine a minimum intensity of pulse stimulation needed to elicit a conscious sensation of feeling in the patient through a process of applying one or more series of pulse signals to the patient, with each series of pulse signals encompassing pulse signals applied at differing intensity levels. The patient then indicates how many pulse signals were consciously felt (by providing appropriate user input), and this data is utilized to determine the minimum intensity of pulse signals needed to elicit a conscious feeling within the patient. These processes are performed for one or more discrete regions of the patient's body (each discrete region corresponding to a group of nerve endings tested as a cohesive unit) using a transmitter apparatus comprising a plurality of individual transmitters placed to stimulate specific regions of the patient's body.

For determining other aspects of the peripheral neuropathy of the patient, specifically for determining a nerve velocity of the patient, a pulse signal is applied to a particular region of the patient's body under test, and a timestamp is recorded indicative of the moment the pulse signal was applied. A separate receiver apparatus monitors a nerve at a test distance away from the nerve endings under test and records a timestamp when a body-signal corresponding to the applied pulse signal passes through a test region of the monitored nerve, as detected by the receivers of the receiver apparatus. Using the difference in timestamps and the test distance, a nerve velocity is calculated corresponding to the tested nerve endings. The results of these tests may be recorded over time to determine a progression of peripheral neuropathy for the patient, over time.

Certain embodiments are directed to a system for mapping peripheral neuropathy, the system comprising: a transmitter apparatus comprising a plurality of signal transmitters each configured to emit a nerve-detectable signal to a patient at a known location on the patient; a receiver apparatus comprising a plurality of receivers for detecting body-signals passing along a nerve of the patient; a computing entity in communication with the transmitter apparatus and the receiver apparatus, wherein the computing entity is configured to: cause the transmitter apparatus to emit at least one nerve-detectable signal via a first signal transmitter of the plurality of signal transmitters at an emit timestamp; receive, from the receiver apparatus, data identifying a detect timestamp indicating detection of a body-signal; determine a nerve velocity based at least in part on the emit timestamp and the detect timestamp; and generate a user interface comprising display data generated based at least in part on the nerve velocity for display via a display device.

In certain embodiments, the computing entity is configured to cause the transmitter apparatus to emit a series of nerve-detectable signals via the first signal transmitter, wherein the series of nerve-detectable signals comprise a plurality of nerve-detectable signals having differing intensities. According to certain embodiments, the computing entity is additionally configured to receive patient data identifying a number of signals subjectively felt by the patient. In various embodiments, the computing entity is additionally configured to: cause the transmitter apparatus to emit at least one nerve-detectable signal via a second signal transmitter of the plurality of signal transmitters at a second emit timestamp, wherein the second signal transmitter is located at a different location on the receiver apparatus than the first signal transmitter; receive, from the receiver apparatus, data identifying a second detect timestamp indicating detection of a second body-signal; and determine a nerve velocity corresponding with the second signal transmitter based at least in part on the second emit timestamp and the second detect timestamp.

In certain embodiments, generating the user interface comprises generating a graphical map of nerve velocities corresponding with the first signal transmitter and the second signal transmitter. In various embodiments, the nerve-detectable signal is embodied as one of: an electrical pulse, a vibration, or a heat signal. In certain embodiments, the transmitter apparatus is a wearable device contouring to a patient's foot and the receiver apparatus is a wearable device contouring to the patient's knee. In various embodiments, the computing entity is in wireless communication with the transmitter apparatus and the receiver apparatus. In certain embodiments, the computing entity is additionally configured to, when causing the transmitter apparatus to emit the at least one nerve-detectable signal, record a body position indication identifying a position of the patient's body at the emit timestamp; and wherein generating a user interface comprises displaying the body position indication together with the data generated based at least in part on the nerve velocity.

Various embodiments are directed to a method for mapping peripheral neuropathy, the method comprising: causing a first signal transmitter of a plurality of signal transmitters within a transmitter apparatus to emit at least one nerve-detectable signal at an emit timestamp to a known location on a patient; receiving, from a receiver apparatus comprising a plurality of receivers for detecting body-signals passed along a nerve of the patient, data identifying a detect timestamp indicating detection of a body-signal; determining, a nerve velocity based at least in part on the emit timestamp and the detect timestamp; and generating a user interface comprising display data generated based at least in part on the nerve velocity for display via a display device.

In various embodiments, the method further comprises causing the transmitter apparatus to emit a series of nerve-detectable signals via the first signal transmitter, wherein the series of nerve-detectable signals comprise a plurality of nerve-detectable signals having differing intensities. In certain embodiments, the method further comprises receiving patient data identifying a number of signals subjectively felt by the patient. In certain embodiments, the method additionally comprises: causing the transmitter apparatus to emit at least one nerve-detectable signal via a second signal transmitter of the plurality of signal transmitters at a second emit timestamp, wherein the second signal transmitter is located at a different location on the receiver apparatus than the first signal transmitter; receiving, from the receiver apparatus, data identifying a second detect timestamp indicating detection of a second body-signal; and determining a nerve velocity corresponding with the second signal transmitter based at least in part on the second emit timestamp and the second detect timestamp.

In certain embodiments, generating the user interface comprises generating a graphical map of nerve velocities corresponding with the first signal transmitter and the second signal transmitter. In various embodiments, the nerve-detectable signal is embodied as one of: an electrical pulse, a vibration, or a heat signal. In certain embodiments, the method further comprises: when causing the transmitter apparatus to emit the at least one nerve-detectable signal, recording a body position indication identifying a position of the patient's body at the emit timestamp; and wherein generating a user interface comprises displaying the body position indication together with the data generated based at least in part on the nerve velocity.

Certain embodiments are directed to a computer program product for automated call-analysis, the computer program product comprising at least one non-transitory computer-readable storage medium having computer-readable program code portions stored therein, the computer-readable program code portions configured to: cause a first signal transmitter of a plurality of signal transmitters within a transmitter apparatus to emit at least one nerve-detectable signal at an emit timestamp to a known location on a patient; receive, from a receiver apparatus comprising a plurality of receivers for detecting body-signals passed along a nerve of the patient, data identifying a detect timestamp indicating detection of a body-signal; determine, a nerve velocity based at least in part on the emit timestamp and the detect timestamp; and generate a user interface comprising display data generated based at least in part on the nerve velocity for display via a display device.

In various embodiments, the one or more processors are further configured to cause the transmitter apparatus to emit a series of nerve-detectable signals via the first signal transmitter, wherein the series of nerve-detectable signals comprise a plurality of nerve-detectable signals having differing intensities. In certain embodiments, the one or more processors are further configured to: cause the transmitter apparatus to emit at least one nerve-detectable signal via a second signal transmitter of the plurality of signal transmitters at a second emit timestamp, wherein the second signal transmitter is located at a different location on the receiver apparatus than the first signal transmitter; receive, from the receiver apparatus, data identifying a second detect timestamp indicating detection of a second body-signal; and determine a nerve velocity corresponding with the second signal transmitter based at least in part on the second emit timestamp and the second detect timestamp.

In certain embodiments, the one or more processors are further configured to: when causing the transmitter apparatus to emit the at least one nerve-detectable signal, record a body position indication identifying a position of the patient's body at the emit timestamp; and wherein generating a user interface comprises displaying the body position indication together with the data generated based at least in part on the nerve velocity.

### BRIEF DESCRIPTION OF THE SEVERAL VIEWS OF THE DRAWINGS

Reference will now be made to the accompanying drawings, which are not necessarily drawn to scale, and wherein:

FIG. 1 is an exemplary overview of a system architecture that can be used to practice various embodiments;

FIG. 2 is an example schematic of a management computing entity in accordance with certain embodiments;

FIG. 3 is an example schematic of a user computing entity in accordance with certain embodiments;

FIGS. 4A-4B are illustrations of nerve systems within a human body that may be analyzed in accordance with certain embodiments;

FIGS. 5A-5C are example transmitter apparatuses according to certain embodiments;

FIG. 6 is an example receiver apparatus according to certain embodiments; and

FIG. 7 is a flowchart illustrating example processes of operation of the devices described in accordance with certain embodiments.

### DETAILED DESCRIPTION

The present disclosure more fully describes various embodiments with reference to the accompanying drawings. It should be understood that some, but not all embodiments are shown and described herein. Indeed, the embodiments may take many different forms, and accordingly this disclosure should not be construed as limited to the embodiments set forth herein. Rather, these embodiments are provided so that this disclosure will satisfy applicable legal requirements. Like numbers refer to like elements throughout.

### Overview

To objectively characterize peripheral neuropathy experienced by patients, testing methodologies are provided for providing repeatable tests of aspects of the patient's nervous system functionality—such as the ability of nerve endings to generate consciously detectable sensations of feeling for the patient at increasing levels of stimulation of those nerve endings; as well as the velocity at which a patient's nerves transmit signals of sensation from specific collections of nerve endings to the patient's central nervous system within the patient's spinal column.

A determination of the patient's ability to consciously detect sensations within discrete regions of the patient's body is performed by applying one or more series of pulse signals of differing intensities to a specific region of the patient's body under test via one or more transmitters within a transmitter apparatus. After each series of pulses, the patient indicates how many pulses were actually felt, providing an indication of a minimum level of intensity necessary to elicit a conscious sensation within the patient. This process may be repeated to more precisely determine the minimum level of intensity necessary to elicit a conscious sensation of the patient. This process may additionally be repeated at multiple iterations (e.g., on different days) over an extended time period, to provide an indication of the progression of peripheral neuropathy over time.

A determination of the changes in nerve velocity over time may additionally be detected utilizing the transmitter apparatus and a separate receiver apparatus monitoring body signals passing along a nerve between collections of nerve endings and a central nervous system of the patient. Pulse signals are applied to specific collections of nerve endings and a precise timestamp is generated indicative of the moment each of these pulse signals are applied. Shortly thereafter, the receiver apparatus detects a body signal passing along a monitored nerve that corresponds to the pulse signal(s) applied to the nerve endings. A timestamp indicative of the moment the relevant body signal is detected is recorded. The nerve velocity is then calculated based at least in part on the difference between the recorded timestamps and the test distance between the nerve endings under test and the location where the body-signals are monitored.

A progression of the various monitored attributes of peripheral neuropathy may be recorded through the generation of temporal and/or spatial models reflecting the peripheral neuropathy of the patient. The spatial models indicate levels of peripheral neuropathy experienced at each of a plurality of discrete locations during a single test, and the temporal models indicate levels of change in peripheral neuropathy experienced at specific locations on the patient's body. The data of these spatial and/or temporal models may be presented via graphical user interfaces, such as a graphical map of the patient's body including indications of objective indicia of peripheral neuropathy experienced by the patient.

### Technical Problem

Peripheral neuropathy can drastically impact the lives of individuals affected by it, but the nature of peripheral neuropathy makes it difficult to accurately and objectively classify the degree to which a particular individual is affected by it. Information about the level of peripheral neuropathy experienced by a particular patient has historically been obtained through discussions with those patients while stimulating the patient's nerve endings with precisely-placed needles. However, it is generally difficult to precisely reproduce tests of a level of peripheral neuropathy experienced by a patient, and therefore it is difficult or impossible to accurately track the progression of peripheral neuropathy over time.

### Technical Solution

To address the technical challenges associated with tracking the progression of peripheral neuropathy over time, embodiments as discussed herein utilize automatically controlled devices for applying nerve stimulation to individual collections of nerve endings in a highly reproducible manner and with corresponding levels of stimulation (e.g., a unit associated with a level of stimulation applied) that may be utilized to objectively characterize a determined minimum level of intensity of stimulation required to elicit a conscious response from the patient. Other objective indicia of peripheral neuropathy are monitored and tracked as well, such as a nerve velocity indicative of the velocity at which a patient's nerves pass signals within the patient's body. These tests result in objective results data that may be easily tracked over time and/or compared between patients, with minimal reliance on more subjective attributes of the patient's ability to accurately characterize feelings within his/her own body.

### Computer Program Products, Methods, and Computing Devices

Embodiments of the present invention may be implemented in various ways, including as computer program products that comprise articles of manufacture. Such computer program products may include one or more software components including, for example, software objects, methods, data structures, and/or the like. A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all non-transitory computer-readable media (including volatile and non-volatile media).

In one embodiment, a non-volatile computer-readable storage medium may include a floppy disk, flexible disk, hard disk, solid-state storage (SSS) (e.g., a solid state drive (SSD), solid state card (SSC), solid state module (SSM), enterprise flash drive, magnetic tape, or any other non-transitory magnetic medium, and/or the like). A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In one embodiment, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present invention may also be implemented as methods, apparatus, systems, computing devices, computing entities, and/or the like. As such, embodiments of the present invention may take the form of a data structure, apparatus, system, computing device, computing entity, and/or the like executing instructions stored on a computer-readable storage medium to perform certain steps or operations. Thus, embodiments of the present invention may also take the form of an entirely hardware embodiment, an entirely computer program product embodiment, and/or an embodiment that comprises combination of computer program products and hardware performing certain steps or operations.

Embodiments of the present invention are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatus, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some exemplary embodiments, retrieval, loading, and/or execution may be performed in parallel such that multiple instructions are retrieved, loaded, and/or executed together. Thus, such embodiments can produce specifically-configured machines performing the steps or operations specified in the block diagrams and flowchart illustrations. Accordingly, the block diagrams and flowchart illustrations support various combinations of embodiments for performing the specified instructions, operations, or steps.

### Exemplary System Architecture

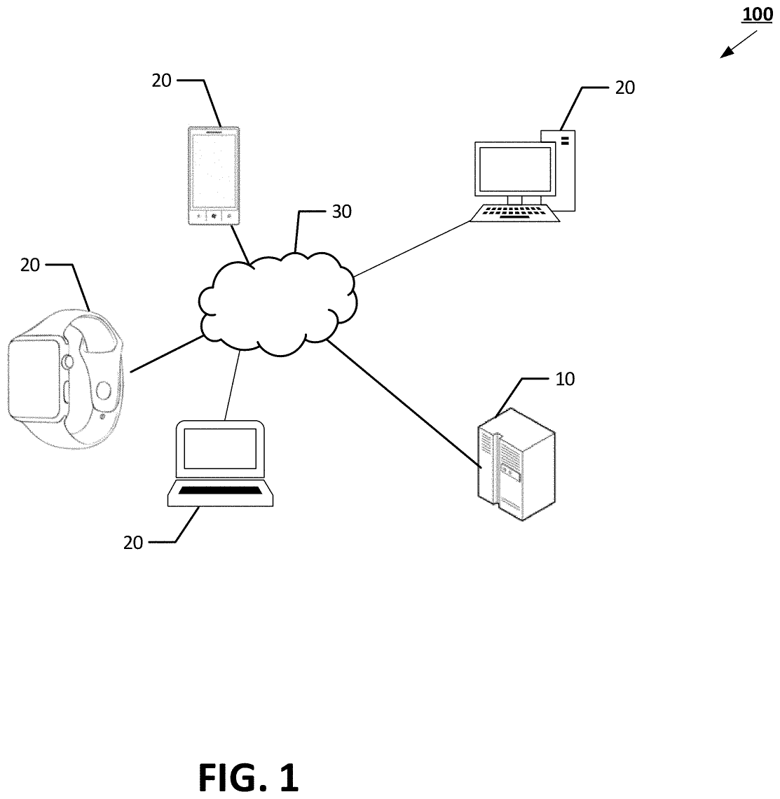

FIG. 1 provides an example system architecture 100 that can be used in conjunction with various embodiments of the present invention. As shown in FIG. 1, the system architecture 100 may comprise one or more management computing entities 10, one or more user computing entities 20, one or more networks 30, and/or the like. Each of the components of the system may be in electronic communication with, for example, one another over the same or different wireless or wired networks 30 including, for example, a wired or wireless Personal Area Network (PAN), Local Area Network (LAN), Metropolitan Area Network (MAN), Wide Area Network (WAN), and/or the like. Additionally, while FIG. 1 illustrates certain system devices as separate, standalone devices, the various embodiments are not limited to this particular architecture.

### Exemplary Management Computing Entity

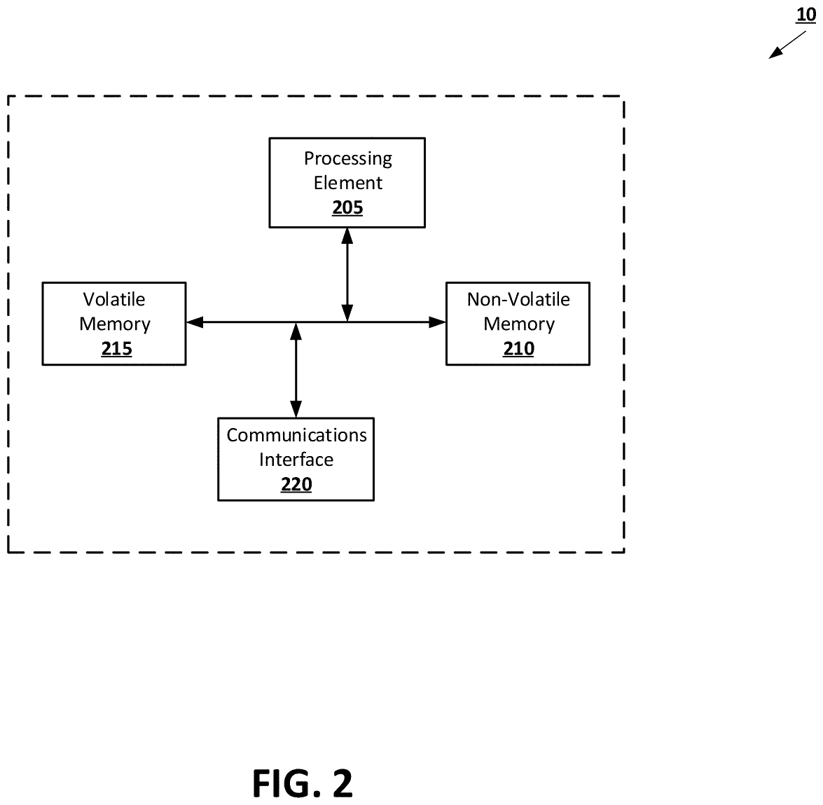

FIG. 2 provides a schematic of a management computing entity 10 according to one embodiment of the present invention. In general, the terms computing device, entity, device, system, and/or similar words used herein interchangeably may refer to, for example, one or more computers, computing devices, computing entities, desktop computers, mobile phones, tablets, phablets, notebooks, laptops, distributed systems, terminals, servers or server networks, blades, gateways, switches, processing devices, set-top boxes, relays, routers, network access points, base stations, the like, and/or any combination of devices adapted to perform the functions, operations, and/or processes described herein. Such functions, operations, and/or processes may include, for example, transmitting, receiving, operating on, processing, displaying, storing, determining, generating/creating, monitoring, evaluating, comparing, and/or similar terms used herein interchangeably. In one embodiment, these functions, operations, and/or processes can be performed on data, content, information, and/or similar terms used herein interchangeably.

As indicated, in one embodiment, the management computing entity 10 may also include one or more network and/or communications interfaces 220 for communicating with various computing entities, such as by communicating data, content, information, and/or similar terms used herein interchangeably that can be transmitted, received, operated on, processed, displayed, stored, and/or the like.

As shown in FIG. 2, in one embodiment, the management computing entity 10 may include or be in communication with one or more processing elements 205 (also referred to as processors, processing circuitry, and/or similar terms used herein interchangeably) that communicate with other elements within the management computing entity 10 via a bus, for example. As will be understood, the processing element 205 may be embodied in a number of different ways. For example, the processing element 205 may be embodied as one or more complex programmable logic devices (CPLDs), microprocessors, multi-core processors, coprocessing devices, application-specific instruction-set processors (ASIPs), and/or controllers. Further, the processing element 205 may be embodied as one or more other processing devices or circuitry. The term circuitry may refer to an entirely hardware embodiment or a combination of hardware and computer program products. Thus, the processing element 205 may be embodied as integrated circuits, application specific integrated circuits (ASICs), field programmable gate arrays (FPGAs), programmable logic arrays (PLAs), hardware accelerators, other circuitry, and/or the like. As will therefore be understood, the processing element 205 may be configured for a particular use or configured to execute instructions stored in volatile or non-volatile media or otherwise accessible to the processing element 205. As such, whether configured by hardware or computer program products, or by a combination thereof, the processing element 205 may be capable of performing steps or operations according to embodiments of the present invention when configured accordingly.

In one embodiment, the management computing entity 10 may further include or be in communication with non-volatile media (also referred to as non-volatile storage, memory, memory storage, memory circuitry and/or similar terms used herein interchangeably). In one embodiment, the non-volatile storage or memory may include one or more non-volatile storage or memory media 210 as described above, such as hard disks, ROM, PROM, EPROM, EEPROM, flash memory, MMCs, SD memory cards, Memory Sticks, CBRAM, PRAM, FeRAM, RRAM, SONOS, racetrack memory, and/or the like. As will be recognized, the non-volatile storage or memory media may store databases, database instances, database management system entities, data, applications, programs, program modules, scripts, source code, object code, byte code, compiled code, interpreted code, machine code, executable instructions, and/or the like. The term database, database instance, database management system entity, and/or similar terms used herein interchangeably may refer to a structured collection of records or information/data that is stored in a computer-readable storage medium, such as via a relational database, hierarchical database, and/or network database.

In one embodiment, the management computing entity 10 may further include or be in communication with volatile media (also referred to as volatile storage, memory, memory storage, memory circuitry and/or similar terms used herein interchangeably). In one embodiment, the volatile storage or memory may also include one or more volatile storage or memory media 215 as described above, such as RAM, DRAM, SRAM, FPM DRAM, EDO DRAM, SDRAM, DDR SDRAM, DDR2 SDRAM, DDR3 SDRAM, RDRAM, RIMM, DIMM, SIMM, VRAM, cache memory, register memory, and/or the like. As will be recognized, the volatile storage or memory media may be used to store at least portions of the databases, database instances, database management system entities, data, applications, programs, program modules, scripts, source code, object code, byte code, compiled code, interpreted code, machine code, executable instructions, and/or the like being executed by, for example, the processing element 205. Thus, the databases, database instances, database management system entities, data, applications, programs, program modules, scripts, source code, object code, byte code, compiled code, interpreted code, machine code, executable instructions, and/or the like may be used to control certain aspects of the operation of the management computing entity 10 with the assistance of the processing element 205 and the operating system.

As indicated, in one embodiment, the management computing entity 10 may also include one or more network and/or communications interfaces 220 for communicating with various computing entities, such as by communicating data, content, information, and/or similar terms used herein interchangeably that can be transmitted, received, operated on, processed, displayed, stored, and/or the like. Such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, management computing entity 10 may be configured to communicate via wireless external communication networks using any of a variety of protocols, such as general packet radio service (GPRS), Universal Mobile Telecommunications System (UMTS), Code Division Multiple Access 200 (CDMA200), CDMA200 1X (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), IR protocols, NFC protocols, RFID protocols, IR protocols, ZigBee protocols, Z-Wave protocols, 6LoWPAN protocols, Wibree, Bluetooth protocols, wireless universal serial bus (USB) protocols, and/or any other wireless protocol. The management computing entity 10 may use such protocols and standards to communicate using Border Gateway Protocol (BGP), Dynamic Host Configuration Protocol (DHCP), Domain Name System (DNS), File Transfer Protocol (FTP), Hypertext Transfer Protocol (HTTP), HTTP over TLS/SSL/Secure, Internet Message Access Protocol (IMAP), Network Time Protocol (NTP), Simple Mail Transfer Protocol (SMTP), Telnet, Transport Layer Security (TLS), Secure Sockets Layer (SSL), Internet Protocol (IP), Transmission Control Protocol (TCP), User Datagram Protocol (UDP), Datagram Congestion Control Protocol (DCCP), Stream Control Transmission Protocol (SCTP), HyperText Markup Language (HTML), and/or the like.

As will be appreciated, one or more of the management computing entity's components may be located remotely from other management computing entity 10 components, such as in a distributed system. Furthermore, one or more of the components may be aggregated and additional components performing functions described herein may be included in the management computing entity 10. Thus, the management computing entity 10 can be adapted to accommodate a variety of needs and circumstances, such as including various components described with regard to a mobile application executing on the user computing entity 20, including various input/output interfaces.

### Exemplary User Computing Entity

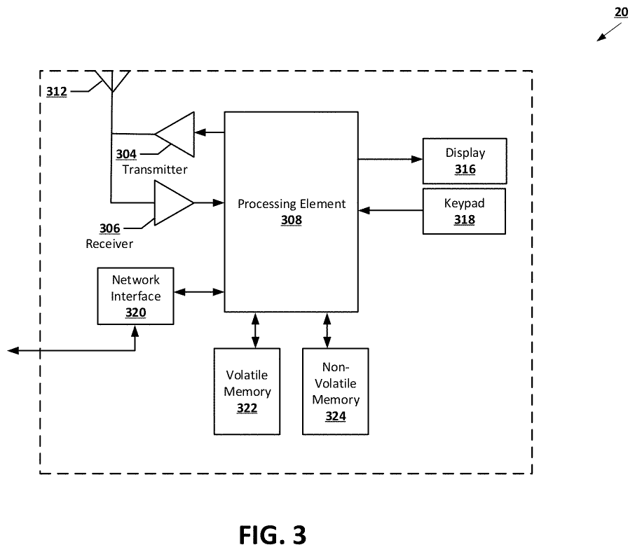

FIG. 3 provides an illustrative schematic representative of user computing entity 20 that can be used in conjunction with embodiments of the present invention. In various embodiments, the user computing entity 20 may be or comprise one or more mobile devices, wearable computing devices, and/or the like.

As shown in FIG. 3, a user computing entity 20 can include an antenna 312, a transmitter 304 (e.g., radio), a receiver 306 (e.g., radio), and a processing element 308 that provides signals to and receives signals from the transmitter 304 and receiver 306, respectively. The signals provided to and received from the transmitter 304 and the receiver 306, respectively, may include signaling information/data in accordance with an air interface standard of applicable wireless systems to communicate with various devices, such as a management computing entity 10, another user computing entity 20, and/or the like. In an example embodiment, the transmitter 304 and/or receiver 306 are configured to communicate via one or more SRC protocols. For example, the transmitter 304 and/or receiver 306 may be configured to transmit and/or receive information/data, transmissions, and/or the like of at least one of Bluetooth protocols, low energy Bluetooth protocols, NFC protocols, RFID protocols, IR protocols, Wi-Fi protocols, ZigBee protocols, Z-Wave protocols, 6LoWPAN protocols, and/or other short range communication protocol. In various embodiments, the antenna 312, transmitter 304, and receiver 306 may be configured to communicate via one or more long range protocols, such as GPRS, UMTS, CDMA200, 1×RTT, WCDMA, GSM, EDGE, TD-SCDMA, LTE, E-UTRAN, EVDO, HSPA, HSDPA, Wi-Fi, Wi-Fi Direct, WiMAX, and/or the like. The user computing entity 20 may also include one or more network and/or communications interfaces 320 for communicating with various computing entities, such as by communicating data, content, information, and/or similar terms used herein interchangeably that can be transmitted, received, operated on, processed, displayed, stored, and/or the like.

In this regard, the user computing entity 20 may be capable of operating with one or more air interface standards, communication protocols, modulation types, and access types. More particularly, the user computing entity 20 may operate in accordance with any of a number of wireless communication standards and protocols. In a particular embodiment, the user computing entity 20 may operate in accordance with multiple wireless communication standards and protocols, such as GPRS, UMTS, CDMA200, 1×RTT, WCDMA, TD-SCDMA, LTE, E-UTRAN, EVDO, HSPA, HSDPA, Wi-Fi, WiMAX, UWB, IR protocols, Bluetooth protocols, USB protocols, and/or any other wireless protocol.

Via these communication standards and protocols, the user computing entity 20 can communicate with various other devices using concepts such as Unstructured Supplementary Service information/data (USSD), Short Message Service (SMS), Multimedia Messaging Service (MMS), Dual-Tone Multi-Frequency Signaling (DTMF), and/or Subscriber Identity Module Dialer (SIM dialer). The user computing entity 20 can also download changes, add-ons, and updates, for instance, to its firmware, software (e.g., including executable instructions, applications, program modules), and operating system.

According to one embodiment, the user computing entity 20 may include location determining aspects, devices, modules, functionalities, and/or similar words used herein interchangeably to acquire location information/data regularly, continuously, or in response to certain triggers. For example, the user computing entity 20 may include outdoor positioning aspects, such as a location module adapted to acquire, for example, latitude, longitude, altitude, geocode, course, direction, heading, speed, UTC, date, and/or various other information/data. In one embodiment, the location module can acquire information/data, sometimes known as ephemeris information/data, by identifying the number of satellites in view and the relative positions of those satellites. The satellites may be a variety of different satellites, including LEO satellite systems, DOD satellite systems, the European Union Galileo positioning systems, the Chinese Compass navigation systems, Indian Regional Navigational satellite systems, and/or the like. Alternatively, the location information/data may be determined by triangulating the apparatus's 30 position in connection with a variety of other systems, including cellular towers, Wi-Fi access points, and/or the like. Similarly, the user computing entity 20 may include indoor positioning aspects, such as a location module adapted to acquire, for example, latitude, longitude, altitude, geocode, course, direction, heading, speed, time, date, and/or various other information/data. Some of the indoor aspects may use various position or location technologies including RFID tags, indoor beacons or transmitters, Wi-Fi access points, cellular towers, nearby computing entities (e.g., smartphones, laptops) and/or the like. For instance, such technologies may include iBeacons, Gimbal proximity beacons, BLE transmitters, NFC transmitters, and/or the like. These indoor positioning aspects can be used in a variety of settings to determine the location of someone or something to within inches or centimeters.

The user computing entity 20 may also comprise a user interface device comprising one or more user input/output interfaces (e.g., a display 316 and/or speaker/speaker driver coupled to a processing element 308 and a touch interface, keyboard, mouse, and/or microphone coupled to a processing element 308). For example, the user interface may be configured to provide a mobile application, browser, interactive user interface, dashboard, webpage, and/or similar words used herein interchangeably executing on and/or accessible via the user computing entity 20 to cause display or audible presentation of information/data and for user interaction therewith via one or more user input interfaces. Moreover, the user interface can comprise or be in communication with any of a number of devices allowing the user computing entity 20 to receive information/data, such as a keypad 318 (hard or soft), a touch display, voice/speech or motion interfaces, scanners, readers, or other input device. In embodiments including a keypad 318, the keypad 318 can include (or cause display of) the conventional numeric (0-9) and related keys (#, *), and other keys used for operating the user computing entity 20 and may include a full set of alphabetic keys or set of keys that may be activated to provide a full set of alphanumeric keys. In addition to providing input, the user input interface can be used, for example, to activate or deactivate certain functions, such as screen savers and/or sleep modes. Through such inputs the user computing entity 20 can capture, collect, store information/data, user interaction/input, and/or the like.

The user computing entity 20 can also include volatile storage or memory 322 and/or non-volatile storage or memory 324, which can be embedded and/or may be removable. For example, the non-volatile memory may be ROM, PROM, EPROM, EEPROM, flash memory, MMCs, SD memory cards, Memory Sticks, CBRAM, PRAM, FeRAM, RRAM, SONOS, racetrack memory, and/or the like. The volatile memory may be RAM, DRAM, SRAM, FPM DRAM, EDO DRAM, SDRAM, DDR SDRAM, DDR2 SDRAM, DDR3 SDRAM, RDRAM, RIMM, DIMM, SIMM, VRAM, cache memory, register memory, and/or the like. The volatile and non-volatile storage or memory can store databases, database instances, database management system entities, information/data, applications, programs, program modules, scripts, source code, object code, byte code, compiled code, interpreted code, machine code, executable instructions, and/or the like to implement the functions of the user computing entity 20.

### Exemplary Networks

In one embodiment, any two or more of the illustrative components of the system architecture 100 of FIG. 1 may be configured to communicate with one another via one or more networks 30. The networks 30 may include, but are not limited to, any one or a combination of different types of suitable communications networks such as, for example, cable networks, public networks (e.g., the Internet), private networks (e.g., frame-relay networks), wireless networks, cellular networks, telephone networks (e.g., a public switched telephone network), or any other suitable private and/or public networks. Further, the networks 30 may have any suitable communication range associated therewith and may include, for example, global networks (e.g., the Internet), MANs, WANs, LANs, or PANs. In addition, the networks 30 may include any type of medium over which network traffic may be carried including, but not limited to, coaxial cable, twisted-pair wire, optical fiber, a hybrid fiber coaxial (HFC) medium, microwave terrestrial transceivers, radio frequency communication mediums, satellite communication mediums, or any combination thereof, as well as a variety of network devices and computing platforms provided by network providers or other entities.

### Example Transmitter Apparatus

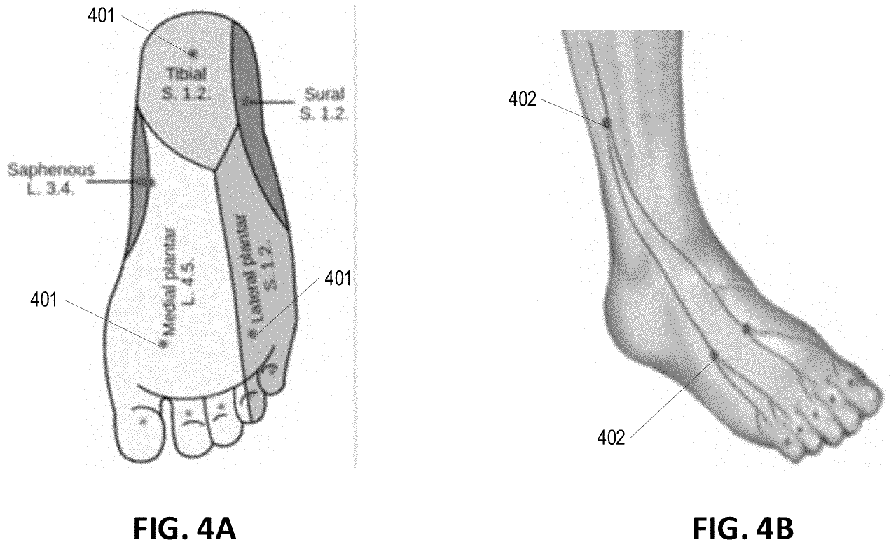

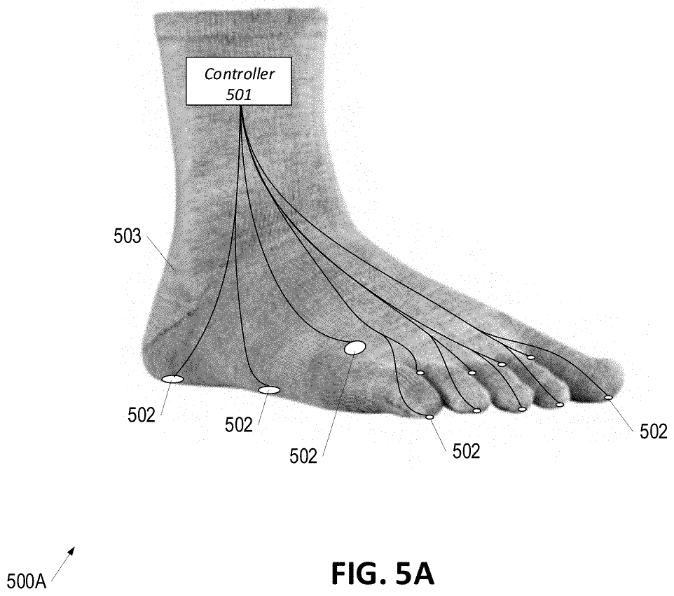

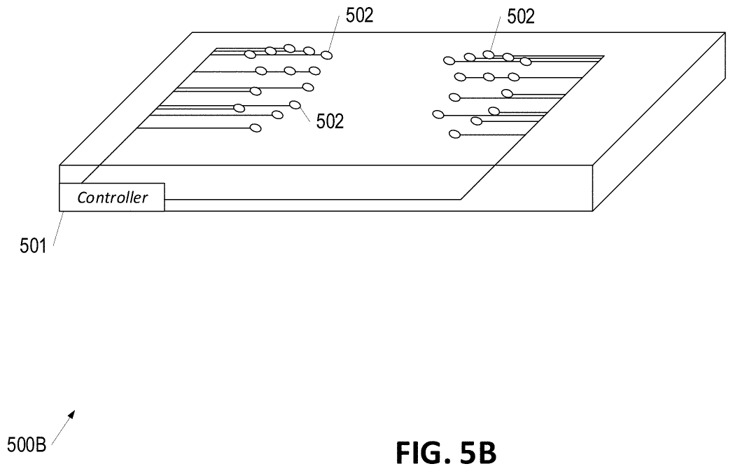

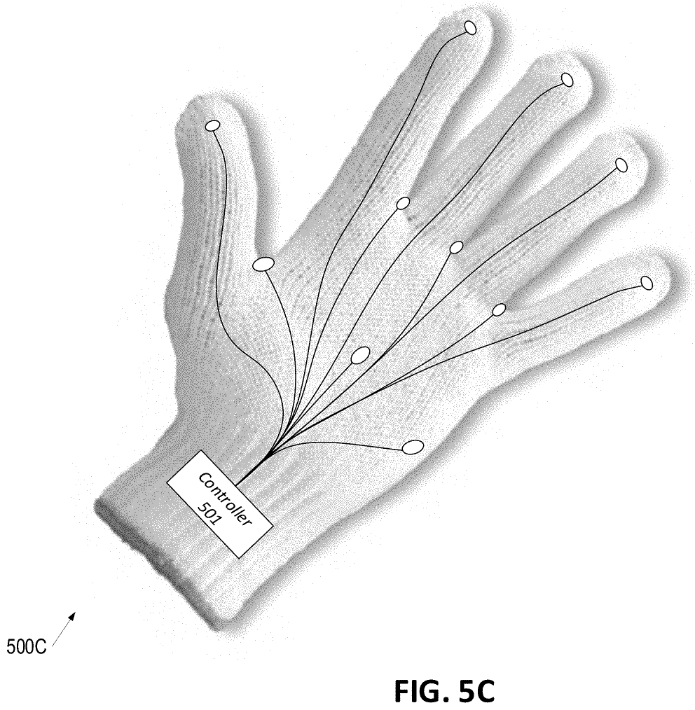

FIGS. 5A-5C illustrate example transmitter apparatuses 500A-C, with FIGS. 4A-4B providing an illustration of nerve systems within a human foot that correlate with the example transmitter apparatus 500A-500B of FIGS. 5A and 5B. Specifically FIG. 5A is a wearable transmitter apparatus 500A (a sock) and FIG. 5B is a non-wearable transmitter apparatus 500B (a platform) configured for providing nerve-detectable signals to the patient's foot. FIG. 5C illustrates an example wearable transmitter apparatus 500C (a glove) for providing nerve-detectable signals to specific regions of a patient's hand.

As shown in the example embodiments of FIGS. 5A-5C, a transmitter apparatus 500A-500C comprises a controller 501 in electronic communication with a plurality of individual transmitters 502 each configured for emitting a nerve-detectable signal and each positioned within a body 503 of the transmitter apparatus 500A-500C. Said differently, the transmitters 502 are each configured for emitting a signal that may be felt by a human user of the transmitter apparatus 500A-500C. As examples, the transmitters 502 may be electrical contacts that are configured to generate a low-voltage shock that may be felt by a patient; vibrators that are configured to generate high-frequency vibrations that may be felt by a patient; resistant heaters that are configured to quickly heat to a high temperature that may be detected by a patient; actuators that are configured to move a plunger, a needle, or another object into the surface of the patient's skin such that the patient can detect the increase in pressure provided by the actuated object; and/or the like. As a specific example, the transmitters 502 may be transcutaneous electrical nerve stimulation (TENS) devices.

In the illustrated example embodiments of FIGS. 5A-5C, the individual transmitters 502 are in wired communication with the controller 501, thereby enabling the controller 501 to provide power signals to the individual transmitters 502 to individually operate the transmitters 502 during operation as discussed below. The controller 501 is configured to operate one or more transmitters 502 simultaneously, such as operating a single transmitter 502 (to emit a nerve-detectable signal from a single transmitter 502 to the patient), or operating a plurality of transmitters 502 simultaneously (to emit a nerve-detectable signal from the plurality of transmitters 502 simultaneously to the patient).

Particularly for wearable transmitter apparatuses 500A, 500C, the transmitters 502 may be low-profile transmitters that, when not in operation for emitting signals, may be undetectable or at least substantially undetectable by the patient. For example, for transmitters located on an underside of the patient's foot when worn in a sock-like wearable transmitter apparatus 500A (with individual regions for enclosing a patient's toes individually so as to enable placement of individual transmitters 502 in the regions between adjacent toes on the patient's foot), the transmitters 502 are sufficiently thin that the patient cannot easily distinguish between the thickness of the sock and the thickness of the transmitters 502.

Moreover, wearable transmitter apparatuses 500A, 500C may have a body 503 configured to contour to a specific portion of a human body (or to the shape of another living patient). As shown, the transmitter apparatus 500A is configured to contour to a human foot and the transmitter apparatus 500C is configured to contour to a human hand. The body 503 may comprise a flexible or semi-rigid material. In certain embodiments, portions of the body 503 may comprise a flexible material and other portions of the body 503 may comprise a semi-rigid material. For example, a flexible material may comprise a flexible fabric, a flexible non-woven material, a flexible polymeric material, and/or the like. A flexible material may have stretchable properties. A semi-rigid material may comprise a foam material, a polymeric material that resists bending, and/or the like. Although not shown in the figures, the body 503 may comprise one or more rigid components, such as braces, hinges, and/or the like, to further ensure proper positioning of the wearable transmitter apparatus 500A, 500C.

The wearable transmitter apparatus 500A, 500C may be custom formed for a particular patient, or the wearable transmitter apparatus 500A, 500C may be provided to fit a plurality of patients. The wearable transmitter apparatus 500A, 500C may be provided in a plurality of discrete sizes (e.g., small, medium, large, extra-large) to accommodate a plurality of patients having similar sized features.

For non-wearable transmitter apparatuses (such as transmitter apparatus 500B), the dimensions of the transmitters 502 may be less restricted, as the transmitters may be embedded within a surface of the transmitter apparatus 500B.

The controller 501 of the example embodiments has certain components and/or functionality analogous to a user computing entity 20. In certain embodiments, the controller 501 additionally includes an onboard power-supply, such as a rechargeable battery, a replaceable battery, and/or the like. The controller 501 is configured for wireless communication with other computing entities, such as via short-range wireless communication protocols (e.g., Bluetooth) or longer-range wireless communication protocols (e.g., Wi-Fi). The controller 501 is thus network connectable, such that the controller 501 can connect with the Internet. In certain embodiments, the transmitter apparatus 500A-500C (inclusive of the controller 501) may be embodied as an Internet of Things (IoT) device configured for exchanging data with a remotely located management computing entity 10 via the Internet. In other embodiments, such as those embodiments in which the controller 501 is configured to wirelessly communicate with other computing entities via short-range wireless communication protocols, the transmitter apparatus 500A-500C may communicate indirectly with a management computing entity 10, such as by providing data to a user computing entity 20 in wireless communication with the transmitter apparatus 500A-500C, and causing the user computing entity 20 to transmit the data to the management computing entity 10.

In certain embodiments, the controller 501 may be detachably secured onto/within the transmitter apparatus 500A-500C. Particularly for wearable transmitter apparatuses 500A, 500C, the controller 501 may be removed to facilitate washing of the body 503. In such embodiments, the individual transmitters 502 may be removable from the body 503, or the individual transmitters 502 may be washable, such that the individual transmitters 502 are not damaged if they remain within the body 503 during washing (e.g., water-based and/or soap-based washing). As an example, the controller 501 may include a connector for connecting with a mated connector at an end of a plurality of wires connected to each of the transmitters 502. The connector can be disconnected to enable the controller 501 to be removed from the body 503 of the transmitter apparatus 500A-500C.

With specific reference to the transmitter apparatus 500A of FIG. 5A and the schematic drawings of nerve placement within a human foot of FIGS. 4A-4B, the individual transmitters 502 are positioned within the body 503 of the transmitter apparatus 500A to stimulate individual nerves (or specific families of nerve endings) of the patient. The locations of individual transmitters 502 within a transmitter apparatus 500A may be established and/or optimized through any of a variety of processes, such as through machine-learning based models for selecting effective locations for placement of transmitters 502 within a transmitter apparatus 500A. As shown in the schematic of FIG. 4A, a human foot has a plurality of discrete regions 401 of nerve endings that are believed to correlate to a shared nerve subsystem 402 (as shown in FIG. 4B). Effectively, it is believed that nerve endings within a discrete region 401 communicate with a shared larger nerve 402, such that damage to the larger nerve 402 can be detected by determining whether signals applied within a corresponding region 401 of the patient's foot can be felt by the patient. Additionally, damage to those individual nerve endings can also be detected by applying signals to those nerve endings to determine whether those nerve endings conduct body signals to the patient's central nervous system. The transmitter apparatus 500A thus includes transmitters 502 positioned to interact with each of those discrete regions 401. Although the exact placement of transmitters 502 as illustrated in the embodiment of FIG. 5A should not be interpreted as limiting, the illustration of transmitter apparatus 500A illustrates that a plurality of transmitters 502 may be positioned within a single region of the patient's foot (as can be determined by comparing the location of the regions 401 shown in FIG. 4A relative to the positions of the transmitters 502 shown in FIG. 5A), thereby enabling the transmitter apparatus 500A to provide highly granular signal application to regions of the patient's foot. As a specific example, transmitters 502 may be positioned on the underside of each toe, at the crotch-like region between the toes, on the ball of the patient's foot, on the heel of the patient's foot, on the instep and outstep of the patient's foot, and at one or more locations on the top of the patient's foot. It should be understood that any number of transmitters 502 may be incorporated into the transmitter apparatus 500A, and the number of transmitters 502 may be selected based on a desired granularity in capability of applying nerve-detectable signals to the patient's foot.

A non-wearable transmitter apparatus (e.g., transmitter apparatus 500B) includes a plurality of individual transmitters 502, so as to interact with discrete regions of a patient's body, in a manner analogous to that discussed above in reference to FIGS. 4A-5A. In the example of FIG. 5B, the transmitter apparatus 500B includes a plurality of transmitters 502 positioned to interact with the discrete regions of a patient's foot as discussed in reference to FIG. 4A. The non-wearable transmitter apparatus 500B may include any of a variety of foot-placement assist features, such as printed alignment lines (e.g., directing the patient to place his/her foot within/along the printed alignment lines), contoured features to contour to the patient's foot, and/or the like. The foot-placement assist features (other body parts may be subject to similar alignment features, as needed) facilitate placement of the patient's foot such that the individual transmitters 502 are placed in expected locations relative to the patient's foot.

The examples of FIGS. 5A-5C are provided as non-limiting examples, and it should be understood that other configurations, such as to accommodate other parts of the human body, may be provided in certain embodiments. For example, a wearable or non-wearable transmitter apparatus may be provided for applying nerve-detectable signals to a patient's leg, a patient's arm, a patient's torso, and/or the like.

In certain embodiments, a transmitter apparatus 500A-500C includes one or more alignment or calibration features provided to determine the relative locations of individual transmitters 502 relative to a human body part. Alignment features may be passive features, such as printed/sewn/provided lines on the transmitter apparatus 500A-500C to be aligned with discrete features on the human body (e.g., a line on a sock to be aligned with the front of a patient's shin), a foot-shaped line on a non-wearable transmitter apparatus 500B in which the patient is directed to step, and/or the like. Alignment/calibration features may be mechanically active, such as deformable foam within a generally planar surface that displaces under the weight of a patient's body part to contour to the patient's body part. Transmitters 502 may be repositioned within the deformable foam to be placed within desired positions relative to the patient's body part. Alignment/calibration features may be electronically active, such as capacitive sensors, pressure sensors, and/or the like, that are configured to electronically sense (based on signal outputs of the sensors) the positioning of a human body part, and to map known transmitter locations within the transmitter apparatus to expected locations of individual features of a patient's body part as predicted based at least in part on the sensor output. Electronically active calibration configurations may utilize one or more machine-learning based models, trained using a supervised learning training dataset and associated training methodology based at least in part on historical data of alignment of other historical patients together with user input specifying the specific context of the alignment of those historical patients. Using such a machine-learning based model, the alignment/calibration features detect various features of the human body of the patient and detects the exact alignment of the transmitter apparatus 500 relative to the human body so as to correlate specific transmitters 502 within the transmitter apparatus 500 with specific portions of the human body (e.g., specific sets of nerve endings within the human body).

As an example, a patient may step onto a pressure-sensitive planar transmitter apparatus 500B. By detecting the relative pressure on various locations on the surface of the pressure-sensitive planar transmitter, the transmitter apparatus 500B is able to map the location of the ball of the patient's foot, the patient's individual toes, the patient's heel, the patient's outstep, and/or the like, relative to fixed transmitter 502 positions within the surface of the transmitter apparatus 500B, thereby enabling the controller 501 to control individual transmitters 502 to apply nerve-detectable signals to desired locations of the patient's foot.

Moreover, in certain embodiments, the transmitter apparatus 500A-500C may be configured for generated a plurality of different signal types, such as signals of different frequencies, signals of different intensities, and/or the like. The transmitter apparatus 500A-500C may be operated to select optimal signal types to be used in combination with a particular receiver apparatus 600 (e.g., using machine-learning based modelling to select optimal signals to be used for a patient and with a particular receiver apparatus), so as to optimize performance of the receiver and transmitter combination.

### Example Receiver Apparatus

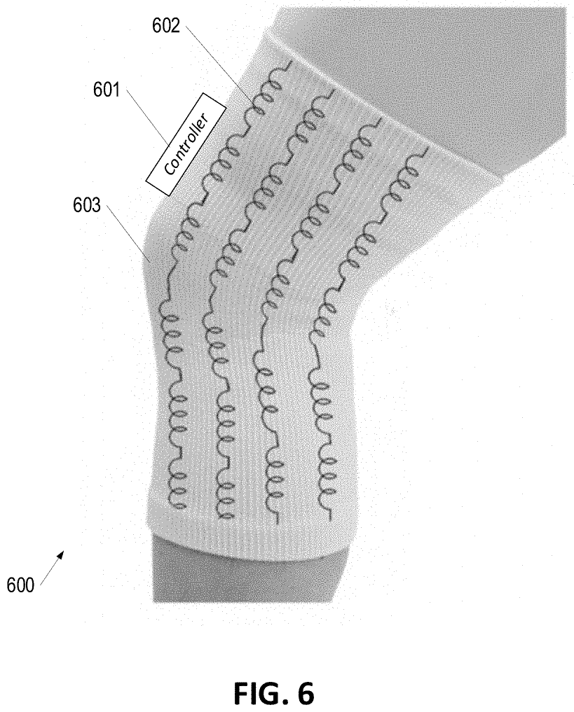

FIG. 6 is a schematic illustration of a wearable receiver apparatus 600 according to one example embodiment. The receiver apparatus 600 of FIG. 6 is a knee-sleeve, however it should be understood that the receiver apparatus 600 may have other configurations to accommodate other body parts, such as an elbow sleeve, a shoulder sleeve, a torso sleeve, a neck sleeve, and/or the like.

As shown in the example embodiment of FIG. 6, a receiver apparatus 600 comprises a controller 601 in electronic communication with a plurality of individual receivers 602 positioned within a body 603 of the receiver apparatus 600. Each of the receivers 602 are highly sensitive electronic signal detectors configured to detect signals passing along nerves within a human body (those signals being referred to herein as “body signals”). The plurality of receivers 602 as illustrated in FIG. 6 encompass a plurality of receivers 602 spaced around a perimeter of the receiver apparatus 600 at an at least substantially even vertical spacing along the height/length of the receiver apparatus 600. Moreover, the plurality of receivers 602 as illustrated in FIG. 6 encompass a plurality of receivers spaced along the height/length of the receiver apparatus 600 so as to expand the detection region of the receiver apparatus 600 to encompass a detection length measured along the height/length of the receiver apparatus 600. In certain embodiments, the plurality of receivers 602 are positioned at respective receiver positions around the perimeter of the receiver apparatus 600, and a plurality of receivers are positioned at each receiver position, extending along the detection length. It should be understood that the positioning of receivers 602 within the receiver apparatus 600 may be optimized and/or otherwise selected via any of a variety of methodologies, including machine-learning based models for optimizing the positioning of the receivers 602 to optimally monitor a particular nerve conduit within the body. As an example, the receivers 602 are inductive detectors comprising highly sensitive coils of fine conductive wire to detect the extremely low-voltage electric signals that travel along the lengths of nerves during normal functionality of a body nervous system. As another example, the receivers 602 may each encompass small-scale RF-style antennas tune to pulse frequencies or signals generated by the transmitters of the transmitter device. As yet another example, the receivers 602 may be nanovolt, low-noise and low-frequency receiver circuits (e.g., which may be used in conjunction with inductive coils) The receivers 602 may operate in an array of a plurality of receivers 602. The receivers 602 of certain example embodiments operate together with one or more amplifier circuits to amplify signals generated upon detection of body-signals detected as passing along a nerve through the detection region. The receivers 602 may operate collectively as a vertically polarized circular antenna system, so as to collectively detect body signals passing along one or more nerves within the region of the human body surrounded by the plurality of receivers 602. In other embodiments, the receivers 602 may be sufficiently sensitive to operate independently or to operate collectively to triangulate the location of detected signals within the human body, such as based on differences in the intensity of signals detected by each individual receiver 602. Thus, in embodiments with extremely high-sensitive receivers 602, the receiver apparatus 600 may facilitate the location of specific nerves within a human body, to provide a three-dimensional map of the location of nerves within the human body based at least in part on the triangulated detected location of body signals within the human body.

In the illustrated example embodiment of FIG. 6, the individual receivers 602 are in wired communication with the controller 601, thereby enabling the receivers 602 to pass signals to the controller 601 indicative of detected body signals during operation, as discussed in greater detail below. The controller 603 is configured to monitor the plurality of individual receivers 602 for signals pushed from one or more individual receivers 602, so as to determine whether the plurality of receivers 602 generate signals indicative of the detection of a body signal passing along a nerve within the human body.

In an effort to minimize the effects of body signals that may be generated within nerves as a result of the placement of the receiver apparatus 600 on the human body, the individual receivers 602 may be low-profile, so as to be minimally detectable by the patient. Because the receivers 602 are provided to detect body signals passing along nerves within a human body, and those body signals are reflective of sensations felt by nerve endings that may be connected to the nerve to be monitored by the individual receivers 602, the physical characteristics of the individual receivers (e.g., weight, size, surface finish, and/or the like) may be provided to minimize sensations felt by the human body and which may create noise signals within the nerve that may interfere with signals desired to be detected (e.g., body signals originating from the transmitter apparatus 500A-500C). As the individual receivers 602 are integrated within the body 603 of the receiver apparatus 600, the individual receivers 602 may be provided within the material of the body 603, such that the patient cannot easily distinguish between the thickness of the body 603 and the thickness of the individual receivers 602.

Moreover, the wearable receiver apparatus 600 comprises a body configured to contour to a specific portion of the human body (or to the shape of another living patient). As shown, the receiver apparatus 600 is configured to contour to a human knee. The body 603 may comprise a flexible or semi-rigid material. In certain embodiments, portions of the body 603 may comprise a flexible material and other portions of the body 603 may comprise a semi-rigid material. As mentioned above, a flexible material may comprise a flexible fabric, a flexible non-woven material, a flexible polymeric material, and/or the like. A semi-rigid material may comprise a foam material, a polymeric material that resists bending, and/or the like. Although not shown in the figures, the body 603 may comprise one or more rigid components, such as braces, hinges, and/or the like, to further ensure proper positioning of the wearable receiver apparatus 600.

The wearable receiver apparatus 600 may be custom formed for a particular patient, or the wearable receiver apparatus 600 may be provided to fit a plurality of patients. The wearable receiver apparatus 600 may be provided in a plurality of discrete sizes (e.g., small, medium, large, extra-large) to accommodate a plurality of patients having similar sized features.

The controller 601 of the example embodiments has certain components and/or functionality analogous to a user computing entity 20. In certain embodiments, the controller 601 additionally includes an onboard power-supply, such as a rechargeable battery, a replaceable battery, and/or the like. The controller 601 is configured for wireless communication with other computing entities, such as via short-range wireless communication protocols (e.g., Bluetooth) or longer-range wireless communication protocols (e.g., Wi-Fi). The controller 601 is thus network connectable, such that the controller 601 can connect with the Internet. In certain embodiments, the receiver apparatus 600 (inclusive of the controller 601) may be embodied as an IoT device configured for exchanging data with a remotely located management computing entity 10 via the Internet. In other embodiments, such as those embodiments in which the controller 601 is configured to wirelessly communicate with other computing entities via short-range wireless communication protocols, the receiver apparatus 600 may communicate indirectly with the management computing entity 10, such as by providing data to a user computing entity 20 in wireless communication with the receiver apparatus 600, and causing the user computing entity 20 to transmit data to the management computing entity 10.

In certain embodiments, the controller 601 may be detachably secured onto/within the receiver apparatus 600. Particularly for wearable receiver apparatuses 600, the controller 601 may be removed to facilitate washing of the body 603. In such embodiments, the individual receivers 602 may be removable from the body 603, or the individual receivers 603 may be washable, such that the individual receivers 602 are not damaged if they remain within the body 603 during washing (e.g., water-based and/or soap-based washing). As an example, the controller 601 may include a connector for connecting with a mated connector at an end of the plurality of wires connected to each of the receivers 602. The connector can be disconnected to enable the controller 601 to be removed from the body 603 of the receiver apparatus 600.

The individual receivers 602 are positioned within the body 603 to provide maximum sensitivity to body signals passing through nerves within the portion of the human body covered with the receiver apparatus 600. For example, the individual receivers 602 may be equally spaced around the perimeter of the receiver apparatus 600. In other embodiments, the individual receivers 602 may be concentrated in a portion of the body 603 positioned adjacent to an expected location of a nerve within the portion of the human body covered with the receiver apparatus 600, when the receiver apparatus 600 is properly positioned relative to the human body part. As discussed above, the individual receivers 602 may be sufficiently sensitive to enable triangulation of individual nerves within the portion of the human body covered by the receiver apparatus 600. In such embodiments, the individual receivers 602 are positioned at known locations within the body 603 and are spaced to enable accurate triangulation of body signals detected by the individual receivers 602.

The example of FIG. 6 is provided as a non-limiting example, and it should be understood that other configurations, such as to accommodate other parts of the human body, may be provided in certain embodiments. For example, a wearable or non-wearable receiver apparatus may be provided for monitoring body-signals passing through nerves within a patient's leg, a patient's arm, a patient's torso (e.g., within the patient's spinal column), within the patient's neck, and/or the like.

In certain embodiments, the receiver apparatus 600 includes one or more alignment or calibration features provided to determine the relative locations of individual receivers 602 relative to a human body part. Alignment features may be passive features, such as printed/sewn/provided lines on the receiver apparatus 600 to be aligned with discrete features on the human body (e.g., a line on a knee-sleeve to be aligned with the front of the patient's shin and thigh). Alignment/calibration features may be mechanically active, such as deformable foam within a generally tubular sleeve to be provided over a patient's knee. The deformable foam displaces to accommodate the shape of the patient's knee. Receivers 502 may be repositioned within the deformable foam to be placed within desired positions relative to the patient's body part. Alignment/calibration features may be electronically active, such as capacitive sensors, pressure sensors, and/or the like, that are configured to electronically sense (e.g., based on signal outputs of the sensors) the positioning of the receiver apparatus 600 relative to a human body part, and to map known receiver locations within the receiver apparatus to expected locations of individual features of a patient's body part as predicted based at least in part on the sensor output. Electronically active calibration configurations may utilize one or more machine-learning based models, trained using a supervised learning training dataset and associated training methodology based at least in part on historical data of alignment of other historical patients together with user input specifying the specific context of the alignment of those historical patients. Using such a machine-learning based model, the alignment/calibration features detect various features of the human body of the patient and detect the exact alignment of the receiver apparatus 600 relative to the human body so as to correlate specific receivers 602 within the receiver apparatus 600 with specific portions of the human body.

As an example, a patient may place a tubular sleeve receiver apparatus 600 over his/her knee. By detecting the relative pressure on various locations on the interior surface of the tubular sleeve, the receiver apparatus 600 may determine the orientation of the receiver apparatus 600 relative to the patient's kneecap, thigh, shin, and/or the like, thereby enabling the controller 601 to map signals received from individual receivers 602 to specific locations around the patient's knee.

### Example System Operation

The operation of a system for monitoring peripheral neuropathy is described in reference to FIGS. 4A-7. The described system provides for objective data collection through multiple methodologies to provide multiple sets of results data that can be collected over time to monitor various attributes of peripheral neuropathy of a patient, thereby providing multiple views of progression of the patient's peripheral neuropathy condition. As discussed herein, the described system enables a determination of a level of intensity that must be applied to particular nerve endings to generate a noticeable sensation to the patient (such that the patient can consciously sense the applied signal). The described system additionally enables a determination of nerve velocity, indicative of the amount of time for an applied signal at a particular set of nerve endings to be passed to a particular location of a nerve as the generated body signal is passed to the central nervous system.

### Monitoring Progression of Conscious Peripheral Neuropathy

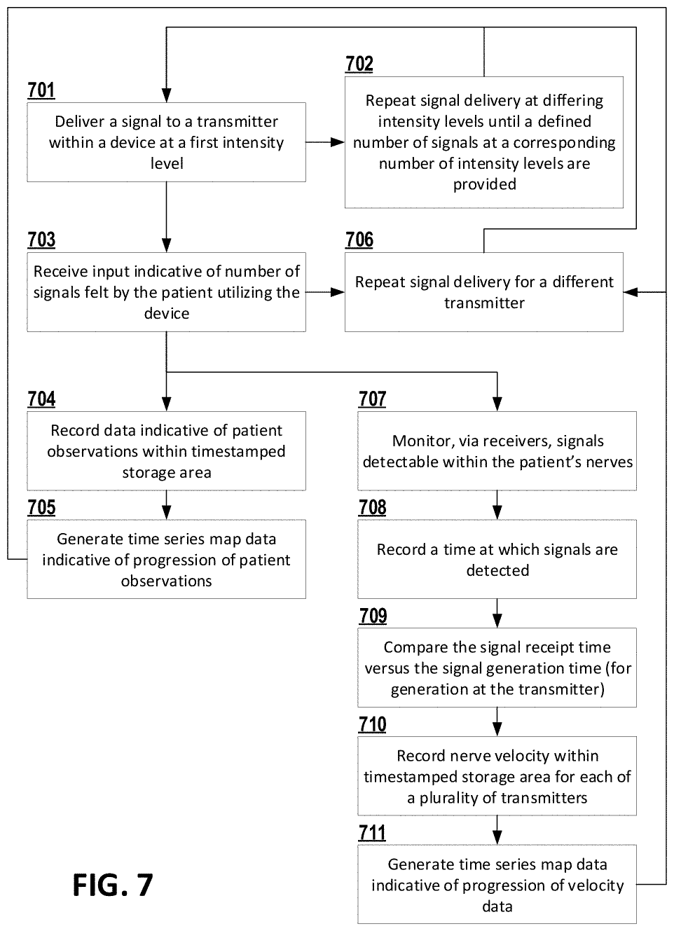

FIG. 7 provides a flowchart illustrating example operations that may be performed by a system and described herein for monitoring peripheral neuropathy of a patient. As mentioned above, an example aspect of monitoring peripheral neuropathy is determining a level of intensity of a signal applied to the patient's body part necessary to be consciously felt by the patient (reflecting a minimum level of intensity of stimulation required to cause the nerve endings at the location of stimulation to conduct a body signal via the nervous system). This determination provides an indication of the ability of the patient's nerves to conduct signals, and specifically indicates whether the patient's nerves are capable of conducting signals indicative of low-intensity stimulation at specific nerve endings. The determination of a level of intensity necessary to generate a conscious sensation of the patient is provided through a process of sequentially applying a series of signal pulses to a specific set of nerve endings on a patient, with those signal pulses being applied at different intensity levels (e.g., at increasing intensity levels between the first signal pulse and the last signal pulse of the series), and asking the patient to say how many pulses were felt from the series. A series of pulses may always be the same number of pulses (e.g., 5), or the number of pulses may vary, such that patients are unable to guess the total number of signal pulses applied. The series of signal pulses may be varied in other ways, such as by changing the order of signal pulses (e.g., starting at a lowest intensity and moving progressively to a highest intensity; starting at a highest intensity and moving progressively to a lowest intensity; randomly applying pulse signals of different intensities within the series, and/or the like) in order to avoid biasing the patient based on a guessed or expected number of pulses.

The intensity of a signal is defined based on the type of transmitter 502 utilized to apply the signal. For electrical pulse generators, the intensity may be defined based on the voltage and/or current applied (e.g., in micro-volts, milli-amps, or other unit as relevant to the operation of the electrical pulse generator). For vibrators, the intensity may be defined based on the level of intensity of the vibration applied (which may be measured based on the amount of electrical current applied across the mechanical vibration element within the vibration generator). For pressure generators, the intensity may be defined based on the amount of displacement of an actuator pressed into the patient's skin (or force applied to a patient's skin) (e.g., a higher displacement into the patient's skin/higher force correlates to a higher intensity).

With reference to FIG. 7, a method for determining a level of intensity necessary to register a conscious sensation for the patient begins by applying the first of a series of signal pulses, as indicated at Block 701. As indicated at Block 702, the application of a pulse signal is repeated until the entire series of pulse signals is applied to the patient (indicated as a loop between Blocks 701-702). As mentioned above, the series of pulses may encompass 5 pulses of different signal intensities. Although any number of pulse signals may be included within a series of pulse signals. In certain embodiments, the number of pulse signals may vary between applications, such that a patient cannot be biased into expecting a certain number of pulse signals. The pulse signals are applied via a transmitter apparatus 500 as described above. The pulse signals are applied to a single set of nerve endings (e.g., within a defined region of a patient's body part, as discussed in reference to the example of FIGS. 4A-4B, above). As will be discussed in greater detail below, each pulse signal is applied while the controller 501 simultaneously records a timestamp indicative of the time the pulse signal was applied. The data of the timestamp may be stored together with data indicative of the intensity of the applied pulse signal, and this data collectively defines pulse data that is provided to the management computing entity 10 and/or a user computing entity 20 in wireless communication with the controller 501. In certain embodiments, the pulse data is provided together with data identifying the patient, such that the data indicative of applied pulses may be stored in a profile corresponding with the patient, thereby enabling generation of a progression of peripheral neuropathy over time, based at least in part on data generated during multiple tests of nerve sensitivity.

With reference again to FIG. 7, the method continues as indicated at Block 703, with the receipt of user input indicative of the number of pulse signals consciously felt by the patient of the series of pulse signals. The user input may be provided directly into a user interface of the controller 501, the user input may be provided into a user interface of a user computing entity 20 in communication with the controller 501, or the user input may be provided to a user computing entity 20 in communication with the management computing entity 10. In any configuration, the data indicative of the user input is matched with the data indicative of the number of pulse signals applied, the intensity of each pulse signal, and the location of the applied pulse signals on the patient's body so as to provide raw data indicative of the level of sensitivity of the nerve endings of the patient in the area of generation of the pulse signals. In certain embodiments, the system may await user input after each pulse signal, and before applying the next consecutive pulse signal. In other embodiments, the system is configured to apply all pulse signals of a series of pulse signals before requesting user input. In certain embodiments, after applying all pulse signals of a series of pulse signals, if the received user input indicates the user did not consciously feel any pulse signals, the system may loop back to Blocks 701-702 and may apply another series of pulse signals, with at least one pulse signal of the second series of pulse signals having an intensity higher than the highest intensity of the initial series of pulse signals. In one embodiment, all pulse signals of the second series of pulse signals may have intensities higher than the highest intensity pulse signal of the initial series of pulse signals.

Based at least in part on a comparison between the user input indicating the number of pulse signals felt and the intensities of each pulse signal within the series of pulse signals applied, the system (e.g., specifically, the management computing entity 10) determines a minimum level of intensity necessary for the patient to consciously detect the pulse signal. For example, the system may operate based on an assumption that the pulse signals consciously felt are the highest intensity number of pulse signals as indicated as being consciously felt by the patient. Thus, the minimum intensity is indicated as being the minimum intensity within the highest intensity number of pulse signals consciously felt by the patient. As a specific example, if 5 pulse signals were applied, and the user input indicates that the patient felt 3 pulses, the lowest and second lowest intensity pulses are assumed to have not been felt, and so the third highest intensity pulse is determined to be the minimum intensity of a pulse felt by the patient. Data indicative of the minimum intensity felt by the patient may be stored together with additional metadata within the patient's profile, such as a date of testing of nerve sensitivity, as well as additional data indicative of the test for nerve sensitivity applied (e.g., intensities that were not felt by the patient, higher intensities felt by the patient, and/or the like), as indicated at Block 704.

In certain embodiments, the system may be configured to execute a confirmation test once the minimum intensity level is established within a series of pulse signals applied. A second series of pulse signals may be applied to the patient, with the minimum intensity of the second series of pulse signals applied being equal to the assumed minimum intensity identified from the initial series of pulse signals applied. User input is collected after applying the series of pulse signals to the patient to determine the number of pulse signals consciously felt by the patient. This process may be reiterated until the user input indicating the number of pulse signals consciously felt matches the number of pulse signals applied in the series of pulse signals applied. The minimum intensity of the last series of pulse signals applied may be designated as the minimum intensity that can be consciously felt by the patient, and data indicative of this minimum intensity may be recorded (together with the date of the test and/or any other metadata applicable) and stored in the patient's profile (as indicated at Block 704).

The process for generating pulse signals within a series of pulse signals, collecting user input indicative of the number of pulse signals consciously felt, and confirming the minimum intensity necessary to generate a consciously felt sensation within the patient, is repeated for any other areas of the patient's body to be tested through generation of pulse signals to be provided to other portions of the human body (e.g., other nerve endings within the human body, that may be tested through the generation of signals via other transmitter(s) 502 within the transmitter apparatus 500, for example, without moving the transmitter apparatus 500 to another portion of the patient's body), as reflected at Block 706. For example, other transmitters 502 within a transmitter apparatus 500 may be utilized for applying signals to nerve endings in other regions of the patient's body, and the process discussed above can be repeated for the additional area of interest. As a specific example with reference to FIG. 4A, if a first set of pulse signals was generated and transmitted to the medial plantar region of the patient's foot until the process discussed herein for determining the patient's sensitivity to nerve stimulation as discussed above is complete, the process may be repeated with a different transmitter(s) 502 for applying pulse signals to the lateral planar region of the patient's foot.

Once all areas of interest have been tested, and data is generated for each area of interest, the generated data is stored and may be referenced later, such as for generating a temporal model of the progression of peripheral neuropathy, as indicated at Block 705 and as discussed in greater detail herein, inclusive of data generated during different tests on different days. As a part of the generation of a temporal model of the progression of peripheral neuropathy, the system (e.g., the management computing entity 10) may generate one or more graphical models, such as a heatmap, illustrating the relative sensitivity of the tested nerve endings to sensations as well as illustrating the change in sensitivity of the tested nerve endings in various locations of the human body over time.

As discussed above, both the transmitter apparatus 500 and the receiver apparatus 600 are non-invasive devices that have minimal impact on the patient's movement. Thus, testing of the level of intensity of pulse signals required to elicit a conscious sensation within a particular set of nerve endings may be performed while the patient is stationary and/or while the patient is moving (e.g., walking, swinging an appendage on which the nerve velocity is tested, or performing any other movement, as prescribed by the user performing the test). Thus, the testing may be performed to identify particular body positions that impact nerve sensation (e.g., a patient may experience a loss of nerve sensation when walking, but not when standing still). For example, contraction of certain muscles may cause a pinched nerve in certain patients (which may cause or exacerbate peripheral neuropathy during certain activities and/or while the patient's body is in a particular position), and therefore testing for peripheral neuropathy during particular movements/body positions can provide additional insight into the patient's condition.

In certain embodiments, the results generated during a particular test are stored together with data indicative of the movement or position of the patient's body when the test was performed. Data indicative of the movement or position of the patient's body when the test was performed may be provided via user input (e.g., user input provided to a user computing entity 20 in communication with the transmitter apparatus 500 and/or the receiver apparatus 600). In other embodiments, data indicative of the movement or position of the patient's body when the test was performed may be generated automatically, such as based at least in part on data generated by one or more position sensors (e.g., proximity sensors detecting a distance between the receiver apparatus 600 and the transmitter apparatus 500; an accelerometer detecting movement/orientation of the receiver apparatus 600 and/or the transmitter apparatus; and/or the like).

Moreover, certain tests may be performed across multiple locations of a patient's body during a single test. For example, pulse signals may be applied sequentially to a randomly selected series of locations on the patient's body (by randomly activating individual transmitters 502 within the transmitter apparatus 500, in series, to apply pulse signals to the patient's body during a series of pulse signals). This spatial-testing regimen may be particularly useful for performing movement-based tests, such as tests of the patient's nervous system functionality during particular activities (e.g., walking). These spatial-testing regimens may be useful for identifying areas of the patient's body that are subject to various degrees of peripheral neuropathy during particular activities. Under such spatial-testing regimens, a series of a known number of pulse signals is applied to the patient's body at randomly selected (but known) locations while the patient is performing a prescribed activity. The patient is then asked how many pulse signals were consciously felt. If the number of pulses reported does not match the number of pulses actually applied, another series of pulse signals may be applied, including pulses applied to a subset of the locations of the first series of pulse signals, as well as a new set of locations. The patient is again asked how many pulse signals were felt. If the number of pulses reported matches the number of pulses actually applied during the second series of pulse signals, it can be assumed that one of the areas tested with the first series of pulse signals but not tested with the second series of pulse signals is subject to peripheral neuropathy. However, if the number of pulses reported does not match the number of pulses actually applied during the second series of pulse signals, it can be assumed that one of the areas tested in both the first and second series of pulse signals is subject to peripheral neuropathy. This process may be repeated for one or more additional series of pulse signals until all areas of the patient's body (that may be tested with the placement of the transmitter apparatus 500) that are subject to peripheral neuropathy are identified.

Although discussed above in reference to the use of user input indicative of a patient's perception of pulse signals consciously felt by the patient, it should be understood that various embodiments may utilize other data collection techniques and sources for determining whether a pulse signal resulted in body signals conducted to the patient's central nervous system. For example, an Electroencephalogram (EEG) may be utilized to detect brain stimulation that results from application of pulse signals to individual collections of nerve endings at various locations of a patient's body. Particular signals generated by the EEG may be correlated with the application of pulse signals to the patient's body, thereby providing a highly objective indication of whether the pulse signals applied to a particular region of the patient's body resulted in body signals conducted to the patient's central nervous system.

### Monitoring Progression of Nerve Velocity

The signals generated and provided to specific nerve endings within a patient's body by the transmitters 502 as discussed above, may additionally be utilized for monitoring nerve velocity within the patient's body. Thus, processes for determining nerve velocity may operate simultaneously with processes for detecting the amount of stimulation needed to generate a consciously detectable sensation within the particular nerve endings under test for the human body (as discussed in reference to Blocks 701-705 of FIG. 7, above). However, it should be understood that separate signals may be generated and provided to particular sets of nerve endings within the human body for testing nerve velocity as compared with testing the level of stimulation needed to generate a consciously detectable sensation within those same nerve endings.

To monitor nerve velocity, a transmitter apparatus 500 and a receiver apparatus 600 are both worn (or used with, for non-wearable implementations) by the patient, with the transmitter apparatus 500 having a transmitter 502 placed adjacent to the region of the human body to be tested, and the receiver apparatus 600 placed adjacent to a portion of a nerve through which body-signals originating at the nerve endings of the portion of the body under test pass when travelling to the spinal column.

As discussed above and as indicated at Block 701, the process begins with the generation of one or more pulse signals via a transmitter 502 to apply a nerve-detectable signal to the particular area of the human body encompassing the nerve endings to be tested. A signal pulse signal may be applied in certain embodiments, or a series of pulse signals (as discussed above) may be applied). In an effort to distinguish between “noisy” body-signals that may be indicative of other sensations felt by nerve endings on connection with a particular nerve monitored by a receiver apparatus 600, a unique series of pulse signals may be applied, having a distinct temporal pattern that, in theory, can be mapped to a temporal pattern of body-signals that may be detected by a receiver apparatus 600 monitoring body-signals passing along a more centralized nerve located between the nerve-endings under test and the patient's spinal column, along a particular branch of the patient's nervous system.

The generation of a pulse signals via a transmitter 502 triggers the recordation of a timestamp reflecting the time at which the pulse signal was generated. The recorded timestamp is highly precise (e.g., recorded with a milli-second level of precision, a nano-second level of precision, and/or the like) so as to record precise differences in time between the time at which a pulse signal is generated and a time at which a corresponding body-signal is detected by the receiver apparatus 600. The timestamp is recorded together with additional data indicative of the generation of the pulse signal (e.g., an intensity, whether the pulse signal was a part of a pattern of generated pulse signals, a date of the test, a patient identifier, and/or the like) within a patient profile, such as within a set of data corresponding with the particular test conducted.

For those embodiments in which nerve velocity is tested simultaneously with (using the same generated signals) detecting the level of stimulation required to generate a consciously detectable sensation, the system (e.g., via a user computing entity 20) receives user input indicative of the number of pulse signals consciously felt by the patient, as indicated at Block 703. However, the processes reflected by Block 703 need not be performed when simply detecting nerve velocity (it also should be understood that, as a practical matter, the receipt of user input, as reflected in Block 703 would likely occur temporally after one or more of the processes reflected in Blocks 707-711).

As indicated at Block 707, after generation and application of the one or more pulse signals to the nerve endings under test, the receiver apparatus 600 monitors the nerve for body-signals indicatives of the applied pulse signals. Upon detection of body signals, the receiver apparatus 600 records a timestamp indicative of the time at which the body signals were received, as indicated at Block 708. The recorded timestamp is highly precise (e.g., recorded with a milli-second level of precision, a nano-second level of precision, and/or the like) so as to record precise differences in time between the time at which a pulse signal is generated and a time at which a corresponding body-signal is detected by the receiver apparatus 600. In certain embodiments, timestamps are recorded for all detected body signals, including noisy signals. The body signals may then be determined (e.g., via machine-learning based models) to be either noisy body signals or body signals corresponding to the applied pulse signals, and the timestamps recorded for the noisy body signals are discarded. In other embodiments, the distinction between noisy body signals and body signals corresponding to the applied pulse signals is determined in real-time, such that timestamps are only applied to those body signals determined to correspond to the applied pulse signals.

Given the highly precise nature of determining a nerve velocity, the positioning of the receiver apparatus 600 is determined with precision relative to the patient's body, and relative to the transmitter apparatus 500. In certain embodiments, a controller of the receiver apparatus 602 may utilize a machine-learning based positioning model to detect the exact positioning of the receiver apparatus 602 relative to detectable locations on a human body (e.g., a knee, contoured shapes of a patient's calf, and/or the like) using the output of positioning sensors to detect the location of the receiver apparatus 602). The positioning model may be patient-specific, such that the location of placement of the receiver apparatus 602 on a particular patient may be replicated or determined to ensure consistency in measurements of nerve velocity. The receiver apparatus 602 may define an elongated distance along which signals may be detected within the receiver apparatus 602, and the controller, using the positioning model, may select a particular portion of the length/distance of the receiver apparatus 602 to be used for detecting signals, so as to ensure the distance between the location of signal detection and the transmitter apparatus 500 remains consistent, even as the patient moves and even between multiple, discrete measurement sessions. For example, to calibrate the positioning of the receiver apparatus 600, the receiver apparatus 600 (via the receivers 602) detect a nerve signal within the patient's body, and then selects a measurement region defined as particular portion of the nerve (e.g., a 1-inch diameter portion, although the size of the region may be optimized via the positioning model) with a determinable location relative to fixed portions of the patient's body (e.g., relative to the patient's knee and/or other detectable features of the patient's body). As the patient moves and as the receiver 600 is taken off and repositioned (e.g., during subsequent measurement sessions), the positioning model may be utilized to locate the same measurement region to be used for consistently measuring a nerve velocity. Using the measurement region, a distance between the nerve endings under test and the location along the nerve where the receiver apparatus 600 monitors for body-signals for recording a timestamp upon detection of a relevant body-signal is estimated. This distance, referred to herein as the test distance, is utilized for determining an estimated nerve velocity based at least in part on the difference between timestamps for the generation and application of a pulse signal by a transmitter 502 of the transmitter apparatus 500 and for the detection of a corresponding body signal by receiver apparatus 600 and the test distance. The test distance may be generated to reflect the precise location of a transmitter 502 used during a particular test, and thus the test distance may vary depending on which transmitter 502 of the plurality of transmitters 502 within the transmitter apparatus 500 are being utilized, even if the transmitter apparatus 500 is not moved between tests of different nerve endings. In certain embodiments, calculation of the test distance may be determined based at least in part on a detected distance between the receiver apparatus 600 and the transmitter apparatus 500, as well as a known location of a precise transmitter 502 within the transmitter apparatus 500 (that may be utilized to adjust the determined distance between the receiver apparatus 600 and the transmitter apparatus 500 to generate an estimate of the test distance).

With reference to Block 709 of FIG. 7, the system (e.g., via a user computing entity 20 or the management computing entity 10 receiving signals from the controllers 501, 601 of the transmitter apparatus 500 and the receiver apparatus 600) compares the timestamps generated by the transmitter apparatus 500 and the receiver apparatus 600, indicative of the time a pulse signal was generated and the time a body-signal corresponding with the generated pulse signal was received, respectively. The system determines a nerve velocity by determining a difference between the identified timestamps and dividing the determined time difference between timestamps by the test distance to generate a value indicative of a nerve velocity corresponding with the tested nerve endings. The determined nerve velocity is recorded, as indicated at Block 710, together with data indicating the nerve endings tested (the nerve endings for which the determined nerve velocity is applicable). Additional data may be stored as well, such as a test date/time, a patient identifier, and/or the like. The determined nerve velocity may be recorded and stored within a patient profile, such as within a data set corresponding to the test performed (e.g., corresponding to the date on which the test was performed).

The data may be recorded in connection with data generated and recorded for other test dates, to enable generation of a temporal map providing an indication of nerve velocity changes over time, for each of a plurality of locations on the patient's body (e.g., each of a plurality of sets of nerve endings), as reflected at Block 711. These temporal maps are discussed in greater detail below. In certain embodiments, the generated temporal map may be a graphical map providing a visual indication of nerve velocity, as detected using pulse signals provided at each of a variety of locations on the human body (each location may be graphically illustrated as a particular region on a graphical map of the human body).

As discussed above, both the transmitter apparatus 500 and the receiver apparatus 600 are non-invasive devices that have minimal impact on the patient's movement. Thus, testing of nerve velocity may be performed while the patient is stationary and/or while the patient is moving (e.g., walking, swinging an appendage on which the nerve velocity is tested, or performing any other movement, as prescribed by the user performing the test). In certain embodiments, the results generated during a particular test are stored together with data indicative of the movement or position of the patient's body when the test was performed. Data indicative of the movement or position of the patient's body when the test was performed may be provided via user input (e.g., user input provided to a user computing entity 20 in communication with the transmitter apparatus 500 and/or the receiver apparatus 600). In other embodiments, data indicative of the movement or position of the patient's body when the test was performed may be generated automatically, such as based at least in part on data generated by one or more position sensors (e.g., proximity sensors detecting a distance between the receiver apparatus 600 and the transmitter apparatus 500; an accelerometer detecting movement/orientation of the receiver apparatus 600 and/or the transmitter apparatus; and/or the like).

### Development of Temporal and Spatial Models for Monitoring Progression of Peripheral Neuropathy

The data generated and/or collected as a part of monitoring the level of intensity of nerve stimulation required to elicit a consciously detectable sensation for a particular set of nerve endings, as well as data generated indicative of nerve velocity as detected for various sets of nerve endings, may be utilized in the generation of a model demonstrating the progression of peripheral neuropathy of the patient over time.

As mentioned above, the various testing procedures outlined in reference to FIG. 7 may be performed multiple times, over an extended period of time (e.g., on multiple days over a period of days, weeks, months, or years). Each iteration of performance of a test may result in the generation of results data that is tagged with the date/time on which the test was performed. The results data from a plurality of test iterations may be stored within or in association with a patient profile, such that the results data from the plurality of test iterations may be aggregated and utilized to generate a cohesive model demonstrating the progression of the tested peripheral neuropathy of the patient, over time.

As an example, a generated model provides a demonstration of temporal changes in the progression of a patient's peripheral neuropathy for a plurality of locations (e.g., discrete locations within discrete regions on the patient's body) by providing a graphical, animated illustration reflecting the temporal and spatial differences in detected peripheral neuropathy of the patient. An example generated model encompasses a graphical map of at least a portion of the human body, segregating discrete regions of the human body (those discrete regions each encompassing nerve endings tested as a cohesive unit), analogous to the illustration of FIG. 4A. Each region of the generated graphical map may be color coded according to a degree of peripheral neuropathy, as reflected within a single set of results data (corresponding to a single test date/time) and the generated graphical map may be animated to change the color of each discrete region within the graphical map to reflect the level of peripheral neuropathy within the region corresponding to different sets of results data (the results generated on different dates/times). Thus, through the animation of the graphical map, a user can easily view changes in peripheral neuropathy over time. In certain embodiments, a first map is generated to reflect the level of intensity of pulse signals necessary to elicit a consciously detectable sensation within the discrete region, and a second map is generated to reflect the nerve velocity, as calculated for a particular set of nerve endings. Although mentioned above specifically in reference to the use of color to distinguish levels of peripheral neuropathy, it should be understood that any of a variety of methodologies may be utilized to visually distinguish data corresponding to different discrete regions of the human body (e.g., including a numeric representation for comparison of levels of peripheral neuropathy).

In certain embodiments, a stagnant, non-animated map may be generated as a part of a generated model. Like the animated map mentioned above, a map of at least a portion of a human body is divided into discrete regions corresponding to groupings of nerve endings tested as a cohesive unit. Each region may be color coded (or otherwise visually distinguished) to reflect a change in peripheral neuropathy over time. For example, as the level of intensity of a pulse signal required to elicit a conscious sensation changes over time for a particular region of the human body, the graphical illustration of that region may be color coded to reflect the degree of change (or the rate of change, over time) of the level of intensity of a pulse signal required to elicit a conscious sensation. Similarly, as a measured nerve velocity changes over time for a particular region, the graphical illustration of that region may be color coded to reflect the degree of change (or the rate of change, over time) of the nerve velocity.

In various embodiments, models may be generated for different body positions, for different body movements, and/or the like. For example, a first model may be generated for the patient to reflect the progression of peripheral neuropathy while sitting, a second model may be generated for the patient to reflect the progression of peripheral neuropathy while walking, and/or the like. By enabling the generation of different models reflecting different movements, positions, activities, and/or the like of the patient, the system provides a wholistic view of the progression of peripheral neuropathy for a patient over time, reflective of the actual experiences of the patient during typical, normal movement and activities of the patient. These multiple models may be reflected through discrete graphical interfaces generated for each model, or the data of multiple models may be integrated into a single, cohesive graphical interface of a stagnant map or animated map providing data from multiple results data sets (e.g., for different dates/times of test, for different body positions/movements, and/or the like). Thus, through these various models, certain embodiments are configured to identify specific body positions that cause/aggravate the peripheral neuropathy symptoms of a patient.

In various embodiments, the generated models for a particular patient may be utilized to assign a formal diagnosis of peripheral neuropathy to the patient, so as to initiate treatments for the patient. One or more machine-learning based models (e.g., trained via supervised machine learning or unsupervised machine learning) may be utilized to identify degrees of peripheral neuropathy (e.g., a maximum nerve velocity measurement; a minimum intensity of pulse signal for generating a consciously detectable sensation in the tested region of the patient's body, and/or the like), rates of change of peripheral neuropathy, and/or the like for assigning a formal diagnosis of peripheral neuropathy. The one or more models generated for a particular patient may be provided as input to the machine-learning based diagnosis models for determining whether to diagnose the patient with peripheral neuropathy, and the output of the diagnosis models may be one or more notifications or other indications as to whether the patient should undergo treatment for peripheral neuropathy. In certain embodiments, the machine-learning based diagnosis models may additionally be trained using data indicative of various treatments that may be performed for certain patients based on the level, type, and/or location of experienced peripheral neuropathy, and the diagnosis model may then output an indication of a recommended course of treatment for those patients diagnosed with peripheral neuropathy. The output of the diagnosis model may be provided within a graphical user interface provided to a user computing entity 20, for example, as a part of a graphical map of the patient's body. The output of the diagnosis model may be provided so as to indicate graphically on a generated graphical map, what portions of the patient's body are subject to the peripheral neuropathy diagnosis, and/or indicating the recommended treatment to be utilized to address the peripheral neuropathy.

The various patient-specific models discussed herein may be further supplemented with additional subjective data collected from the patient, indicative of the types of sensations felt, the level of sensation felt, and/or any other notes the patient provides for providing additional context regarding the patient's perceived symptoms of peripheral neuropathy. For example, this additional data may be provided within a user interface generated for a particular set of test results, and/or this data may be provided via any of a variety of other mechanisms. In certain embodiments, this additional contextual data may be provided to the diagnosis models (e.g., using natural language processing, structured data approaches (e.g., based on a user's selection of one or more defined options for describing sensations), and/or the like), which may be utilized as additional input that may impact a determined diagnosis for a patient.

Moreover, it should be understood that other graphical user interface configurations may be provided for graphically presenting the data of the patient specific models. For example, bar graphs may be provided indicating a minimum intensity of stimulation required to elicit a consciously detectable sensation for the patient and/or indicating the nerve velocity of the patient. These bar graphs may be location-specific and may include data from a plurality of test dates. These bar graphs may be presented with a numerical indication of the level of improvement of the patient or the level of deterioration of the patient (e.g., a percentage increase or decrease of the intensity of stimulation required to elicit a consciously detectable sensation). The bar graphs may be test-specific and may include data collected at each of a plurality of test locations on the human body during a single test. This test-specific interface may provide an indication of how a patient's peripheral neuropathy is progressing across the patient's body.

### CONCLUSION

Many modifications and other embodiments will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

The methodologies and systems discussed herein may be utilized to test the functionality of individuals who do not have symptoms of peripheral neuropathy to establish baseline data indicative of normal functioning of the nervous system of a human at various locations on the human body. This baseline data may comprise data indicative of a typical intensity of stimulation necessary to elicit a consciously detectable sensation in a human for various locations on the human body, for a normal functioning nervous system. Similarly, data indicative of a nerve velocity may be generated for various locations on human body of a patient that is not experiencing peripheral neuropathy symptoms. In certain embodiments, the baseline data may be stored as a part of training data to be utilized when training a diagnosis model as discussed above. The baseline data may be stored together with additional contextual data of the tested patient, such as an age, gender, and/or other identifying data that may be usable for establishing relevant contextual data for application of the baseline data. Accordingly, the baseline data may be utilized to establish relevant models, thresholds, criteria, and/or the like for diagnosis peripheral neuropathy for various individuals having differing relevant contextual data.

That which is claimed:

1. A system for mapping peripheral neuropathy, the system comprising:
a transmitter apparatus comprising a plurality of signal transmitters each configured to emit a nerve-detectable signal to a patient at a known location on the patient;
a receiver apparatus comprising a plurality of receivers for detecting body-signals passing along a nerve of the patient;
a computing entity in communication with the transmitter apparatus and the receiver apparatus, wherein the computing entity is configured to:
cause the transmitter apparatus to emit at least one nerve-detectable signal via a first signal transmitter of the plurality of signal transmitters at an emit timestamp;
receive, from the receiver apparatus, data identifying a detect timestamp indicating detection of a body-signal;
determine a nerve velocity based at least in part on the emit timestamp and the detect timestamp; and
generate a user interface comprising display data generated based at least in part on the nerve velocity for display via a display device.

2. The system for mapping peripheral neuropathy of claim 1, wherein the computing entity is configured to cause the transmitter apparatus to emit a series of nerve-detectable signals via the first signal transmitter, wherein the series of nerve-detectable signals comprise a plurality of nerve-detectable signals having differing intensities.

3. The system for mapping peripheral neuropathy of claim 2, wherein the computing entity is additionally configured to receive patient data identifying a number of signals subjectively felt by the patient.

4. The system for mapping peripheral neuropathy of claim 1, wherein the computing entity is additionally configured to:
cause the transmitter apparatus to emit at least one nerve-detectable signal via a second signal transmitter of the plurality of signal transmitters at a second emit timestamp, wherein the second signal transmitter is located at a different location on the receiver apparatus than the first signal transmitter;
receive, from the receiver apparatus, data identifying a second detect timestamp indicating detection of a second body-signal; and
determine a nerve velocity corresponding with the second signal transmitter based at least in part on the second emit timestamp and the second detect timestamp.

5. The system for mapping peripheral neuropathy of claim 4, wherein generating the user interface comprises generating a graphical map of nerve velocities corresponding with the first signal transmitter and the second signal transmitter.

6. The system for mapping peripheral neuropathy of claim 1, wherein the nerve-detectable signal is embodied as one of: an electrical pulse, a vibration, or a heat signal.

7. The system for mapping peripheral neuropathy of claim 1, wherein the transmitter apparatus is a wearable device contouring to a patient's foot and the receiver apparatus is a wearable device contouring to the patient's knee.

8. The system for mapping peripheral neuropathy of claim 1, wherein the computing entity is in wireless communication with the transmitter apparatus and the receiver apparatus.

9. The system for mapping peripheral neuropathy of claim 1, wherein the computing entity is additionally configured to, when causing the transmitter apparatus to emit the at least one nerve-detectable signal, record a body position indication identifying a position of the patient's body at the emit timestamp; and
wherein generating a user interface comprises displaying the body position indication together with the data generated based at least in part on the nerve velocity.

10. A method for mapping peripheral neuropathy, the method comprising:
causing a first signal transmitter of a plurality of signal transmitters within a transmitter apparatus to emit at least one nerve-detectable signal at an emit timestamp to a known location on a patient;
receiving, from a receiver apparatus comprising a plurality of receivers for detecting body-signals passed along a nerve of the patient, data identifying a detect timestamp indicating detection of a body-signal;
determining, a nerve velocity based at least in part on the emit timestamp and the detect timestamp; and
generating a user interface comprising display data generated based at least in part on the nerve velocity for display via a display device.

11. The method of claim 10, further comprising causing the transmitter apparatus to emit a series of nerve-detectable signals via the first signal transmitter, wherein the series of nerve-detectable signals comprise a plurality of nerve-detectable signals having differing intensities.

12. The method of claim 11, further comprising: receiving patient data identifying a number of signals subjectively felt by the patient.

13. The method of claim 10, further comprising:
causing the transmitter apparatus to emit at least one nerve-detectable signal via a second signal transmitter of the plurality of signal transmitters at a second emit timestamp, wherein the second signal transmitter is located at a different location on the receiver apparatus than the first signal transmitter;
receiving, from the receiver apparatus, data identifying a second detect timestamp indicating detection of a second body-signal; and
determining a nerve velocity corresponding with the second signal transmitter based at least in part on the second emit timestamp and the second detect timestamp.

14. The method of claim 13, wherein generating the user interface comprises generating a graphical map of nerve velocities corresponding with the first signal transmitter and the second signal transmitter.

15. The method of claim 10, wherein the nerve-detectable signal is embodied as one of: an electrical pulse, a vibration, or a heat signal.

16. The method of claim 10, further comprising: when causing the transmitter apparatus to emit the at least one nerve-detectable signal, recording a body position indication identifying a position of the patient's body at the emit timestamp; and
wherein generating a user interface comprises displaying the body position indication together with the data generated based at least in part on the nerve velocity.

17. A computer program product for automated call-analysis, the computer program product comprising at least one non-transitory computer-readable storage medium having computer-readable program code portions stored therein, the computer-readable program code portions configured to:
cause a first signal transmitter of a plurality of signal transmitters within a transmitter apparatus to emit at least one nerve-detectable signal at an emit timestamp to a known location on a patient;
receive, from a receiver apparatus comprising a plurality of receivers for detecting body-signals passed along a nerve of the patient, data identifying a detect timestamp indicating detection of a body-signal;
determine, a nerve velocity based at least in part on the emit timestamp and the detect timestamp; and
generate a user interface comprising display data generated based at least in part on the nerve velocity for display via a display device.

18. The computer program product of claim 17, wherein the one or more processors are further configured to cause the transmitter apparatus to emit a series of nerve-detectable signals via the first signal transmitter, wherein the series of nerve-detectable signals comprise a plurality of nerve-detectable signals having differing intensities.

19. The computer program product of claim 17, wherein the one or more processors are further configured to:
cause the transmitter apparatus to emit at least one nerve-detectable signal via a second signal transmitter of the plurality of signal transmitters at a second emit timestamp, wherein the second signal transmitter is located at a different location on the receiver apparatus than the first signal transmitter;
receive, from the receiver apparatus, data identifying a second detect timestamp indicating detection of a second body-signal; and
determine a nerve velocity corresponding with the second signal transmitter based at least in part on the second emit timestamp and the second detect timestamp.

20. The computer program product of claim 17, wherein the one or more processors are further configured to: when causing the transmitter apparatus to emit the at least one nerve-detectable signal, record a body position indication identifying a position of the patient's body at the emit timestamp; and
wherein generating a user interface comprises displaying the body position indication together with the data generated based at least in part on the nerve velocity.
